SEQUENCE LISTING (3 sequences)

SEQ ID NO: 1 - protein, 13 aa, synthetic construct
  source 1..13: note = SpyTag amino acid sequence
AHIVMVDAYKPTK

SEQ ID NO: 2 - protein, 134 aa, synthetic construct
  source 1..134: note = SpyCatcher amino acid sequence`
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 3 - protein, 5 aa, synthetic construct
LPXTG

(19) United States
(12) Patent Application Publication
(10) Pub. No.: US 2024/0309423 A1
(43) Pub. Date: Sep. 19, 2024

(54) ON-SEQUENCER FLOWCELL REUSE

(71) Applicant: Illumina, Inc., San Diego, CA (US)

(72) Inventors: Jonathan Boutell, Bishops Stortford (GB); Katharina Mueller-Ott, Saffron Walden (GB); Jason Betley, Buntingford (GB); Xiaolin Wu, Cambridge (GB); Wayne George, Haverhill (GB); Pietro Gatti Lafranconi, Cambridge (GB); Andrew Brown, Cambridge (GB)

(73) Assignee: Illumina, Inc., San Diego, CA (US)

(21) Appl. No.: 18/595,944

(22) Filed: Mar. 5, 2024

Related U.S. Application Data
(60) Provisional application No. 63/488,866, filed on Mar. 7, 2023.

(51) Int. Cl. C12Q 1/44; C12Q 1/6806

(52) U.S. Cl. CPC C12Q 1/44; C12Q 1/6806

(57) ABSTRACT

Automated methods conducted in a sequencing flowcell, and kits for reusing a flowcell, are provided herein. In some examples, an automated method conducted in a sequencing flowcell may include, at a surface of the sequencing flowcell coupled to a first moiety, using a reagent to decouple a first complex from the first moiety. In some examples, the first complex may include a second moiety which couples to the first moiety and a polynucleotide coupled to the second moiety. In some examples, the method may further include using a nuclease to polynucleotides in the sequencing flowcell. The method may include, after using the reagent and after using the nuclease, coupling a second complex to the first moiety. The second complex may include a third moiety which couples with the first moiety and an oligonucleotide coupled to the third moiety.

### CROSS-REFERENCE TO RELATED APPLICATIONS

This application claims the benefit of U.S. Provisional Patent Application No. 63/488,866, filed Mar. 7, 2023 and entitled “On-Sequencer Flowcell Reuse,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2565-US.xml”, was created on Feb. 26, 2024 and is 4 KB in size.

### FIELD

This application relates to sequencing flowcells.

### BACKGROUND

A significant amount of academic and corporate time and energy has been invested into using flowcells to sequence polynucleotides. However, such previously known devices, systems, and methods may not necessarily be sufficiently sustainable or cost-effective. For example, the single-use nature of flowcells may increase the cost of sequencing polynucleotides and create waste.

### SUMMARY

Examples herein relate to on-sequencer flowcell reuse.

Some examples herein provide an automated method conducted in a sequencing flowcell. The method may include, at a surface of the sequencing flowcell coupled to a first moiety, using a reagent to decouple a first complex from the first moiety. The first complex may include a second moiety which couples to the first moiety, and a polynucleotide coupled to the second moiety. The method may include using a nuclease to digest polynucleotides in the sequencing flowcell. The method may include, after using the reagent and after using the nuclease, coupling a second complex to the first moiety. The second complex may include a third moiety which couples with the first moiety, and an oligonucleotide coupled to the third moiety.

In some examples, the second moiety is coupled to the first moiety via a first non-covalent bond, and the third moiety is coupled to the first moiety via a second non-covalent bond.

In some examples, the nuclease is used after the reagent. In some examples, the reagent is used after the nuclease. In still other examples, the reagent and the nuclease are used at the same time as one another.

In some examples, the first complex includes a protein with a first active site to which the oligonucleotide is coupled, and a second active site which corresponds to the second moiety.

In some examples, the surface is coupled to the first moiety using operations including reacting a fourth moiety at the surface with a fifth moiety of a molecule including the first moiety. In some examples, the molecule further includes a linker disposed between the fifth moiety and the first moiety.

In some examples, the method further includes, before using the reagent and before using the nuclease, sequencing the polynucleotide. In some examples, the method further includes after coupling the second complex to the first moiety, using the oligonucleotide to amplify a template polynucleotide, and sequencing the amplified template polynucleotide. In some examples, the flowcell is washed after the second complex is coupled to the first moiety, and before the oligonucleotide is used to amplify the template polynucleotide. In some examples, the flowcell is washed after the polynucleotide is sequenced, before using the reagent, before using the nuclease, and before the second complex is coupled to the first moiety. In some examples, the flowcell is washed after the polynucleotide is sequenced, after using the reagent, after using the nuclease, and before the second complex is coupled to the first moiety.

Some examples herein provide a kit for reusing a flowcell including a surface. The kit may include a plurality of complexes. Each of the complexes may include a second moiety which can couple to a first moiety coupled to the flowcell surface, and a polynucleotide coupled to the second moiety. The kit may include a reagent to decouple the first and second moieties from one another. The kit may include at least one nuclease to digest polynucleotides.

Some examples herein provide an automated method conducted in a sequencing flowcell. The method may include, at a surface of the sequencing flowcell coupled to a first moiety to which a polynucleotide is coupled, digesting the polynucleotide using Micrococcal Nuclease (MNase). The method may include, after digesting the polynucleotide, coupling an oligonucleotide to a second moiety coupled to the surface.

In some examples, the polynucleotide is digested without use of a nuclease besides MNase.

In some examples, the polynucleotide is coupled to the first moiety via a first covalent bond, and wherein the oligonucleotide is coupled to the second moiety via a second covalent bond. In some examples, wherein the oligonucleotide is coupled to the second moiety using operations including reacting the second moiety with a third moiety of a molecule that includes the oligonucleotide.

In some examples, the surface is coupled to the first moiety using operations including reacting a fourth moiety at the surface with a fifth moiety of a molecule including the first moiety. In some examples, the molecule further includes a linker disposed between the fifth moiety and the first moiety.

In some examples, the method further includes, before using the MNase, sequencing the polynucleotide. In some examples, the method further includes, after coupling the oligonucleotide to the second moiety, using the oligonucleotide to amplify a template polynucleotide, and sequencing the amplified template polynucleotide. In some examples, the flowcell is washed after the oligonucleotide is coupled to the second moiety, and before the oligonucleotide is used to amplify the template polynucleotide. In some examples, the flowcell is washed after the polynucleotide is sequenced, before using the MNase, and before the oligonucleotide is coupled to the second moiety.

Some examples herein provide a kit for reusing a flowcell including a surface that includes first moieties. The kit may include molecules each including a second moiety and an oligonucleotide coupled to the first moiety. The second moiety may be to react with one of the first moieties to couple the oligonucleotide to the surface. The kit may include Micrococcal Nuclease (MNase) to digest polynucleotides that are sequenced in the flowcell using the oligonucleotides of the molecules.

Some examples herein provide another kit for reusing a flowcell including a surface that includes first moieties. The kit may include first molecules each including a second moiety and a third moiety. The second moiety may be to react with one of the first moieties to couple the first molecule to the surface. The kit may include second molecules each including a fourth moiety and an oligonucleotide coupled to the fourth moiety. The fourth moiety may be to react with one of the third moieties to couple the oligonucleotide to the surface. The kit may include Micrococcal Nuclease (MNase) to digest polynucleotides that are sequenced in the flowcell using the oligonucleotides of the molecules.

Some examples herein provide a kit for reusing a flowcell including a surface that includes first moieties. The kit may include first molecules to convert the first moieties to second moieties. The kit may include second molecules each including a third moiety and a fourth moiety. The third moiety may be to react with one of the second moieties to couple the second molecule to the surface. The kit may include third molecules each including a fifth moiety and an oligonucleotide coupled to the fifth moiety. The fifth moiety may be to react with one of the fourth moieties to couple the oligonucleotide to the surface. The kit may include Micrococcal Nuclease (MNase) to digest polynucleotides that are sequenced in the flowcell using the oligonucleotides of the molecules.

### BRIEF DESCRIPTION OF DRAWINGS

FIG. 1A illustrates a flow of example operations for using and reusing a flowcell, and example structures formed using such operations.

FIG. 1B illustrates a nonlimiting example of elements that may be used in the example of FIG. 1A.

FIG. 2 illustrates a flow chart showing the flow of operations in an example automated method conducted in a sequencing flowcell.

FIG. 3A illustrates another flow of example operations for using and reusing a flowcell, and example structures formed using such operations.

FIG. 3B illustrates another flow of example operations for using and reusing a flowcell, and example structures formed using such operations.

FIG. 4 illustrates a flow chart showing the flow of operations in another example automated method conducted in a sequencing flowcell.

FIGS. 5A-5B illustrate data collected using an example method.

FIGS. 6A-6C illustrate data collected using an example method.

FIGS. 7A-7B illustrate data collected using an example method.

FIG. 8 illustrates data collected using an example method.

### DETAILED DESCRIPTION

Automated methods conducted in a sequencing flowcell, and kits for reusing a flowcell, are provided herein.

In order to help sequencing become more sustainable, the present subject matter is directed to reusing flowcells by applying different regeneration methods. To allow the end user to reuse flowcells without requiring them to learn additional skills, some examples herein provide flowcell reuse which is fully integrated in a sequencing run, with the flowcell staying on-board the instrument and being regenerated either before or after each run. Described herein are methods and kits that focus on the implementation of regeneration steps on-board a sequencing instrument without additional touch points.

First, some terms used herein will be briefly explained. Then, some example methods for reuse conducted in a sequencing flowcell, and example kits for reusing the flowcell, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to +10%, such as less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthiine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP.

Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, terms such as “covalently coupled” or “covalently bonded” refer to the forming of a chemical bond that is characterized by the sharing of pairs of electrons between atoms. For example, a covalently coupled molecule refers to a molecule that forms a chemical bond, as opposed to a non-covalent bond such as electrostatic interaction.

As used herein, the term “complex” is intended to refer to molecules that are coupled together to form a larger structure. In some examples, a complex may include a central molecule, such as a protein, to which a polynucleotide (such as a capture primer) is coupled. The polynucleotide may be coupled to the central molecule via reaction of a first moiety coupled to the polynucleotide, with a second moiety coupled to the central molecule. Such a reaction may form a covalent bond, or may form a noncovalent bond. As described in greater detail below, the central molecule of a complex may be reversibly coupled to a moiety at a surface.

As used herein, the term “linker” is intended to mean a portion of a molecule via which one element is attached to another element. For example, a linker may attach a first reactive moiety to a second reactive moiety. Linkers may be covalent.

### Automated Methods Conducted in a Sequencing Flowcell, and Kits for Reusing a Flowcell

As will be discussed below, some of the disclosed methods involve strategies for reusing a flowcell. One strategy leverages reversible linkages, such as those between biotin and streptavidin. For example, a surface including a first type of moiety (such as biotin) is generated by grafting a molecule containing that moiety onto the surface (e.g., onto the azides of a polymer coating at that surface). Next, a second type of moiety (e.g., a streptavidin-dualbiotin structure) is used to bind surface primers to the first type of moiety (e.g., biotin) on the surface and enable clustering and sequencing. For regeneration of the surface, the reversible linkage interactions (e.g., biotin-streptavidin interactions) are reversed using a reagent (e.g., hot formamide). A nuclease digest ensures that no DNA from one run will be carried over into the following run.

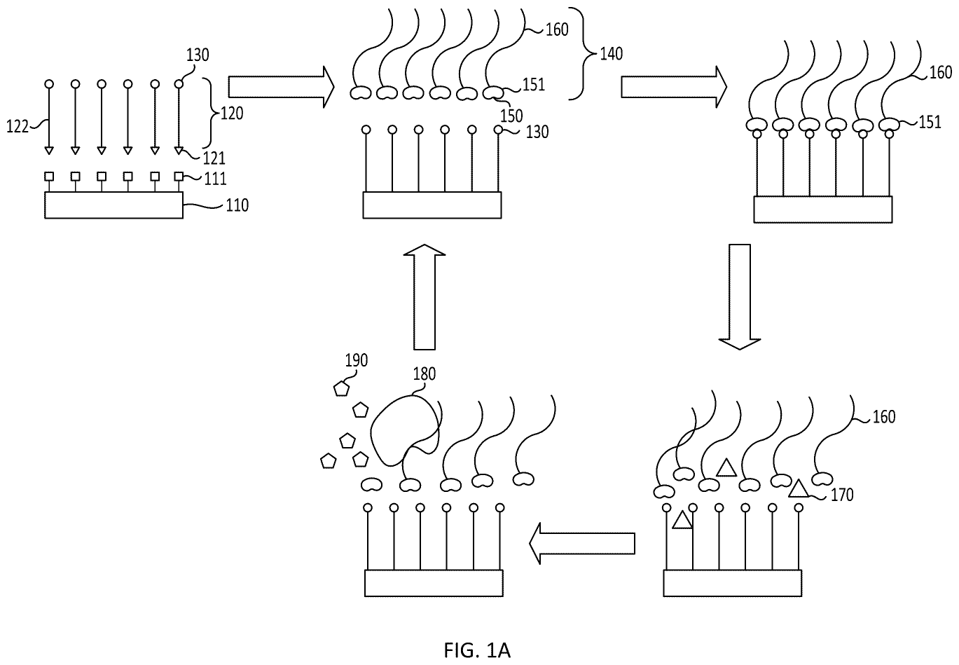

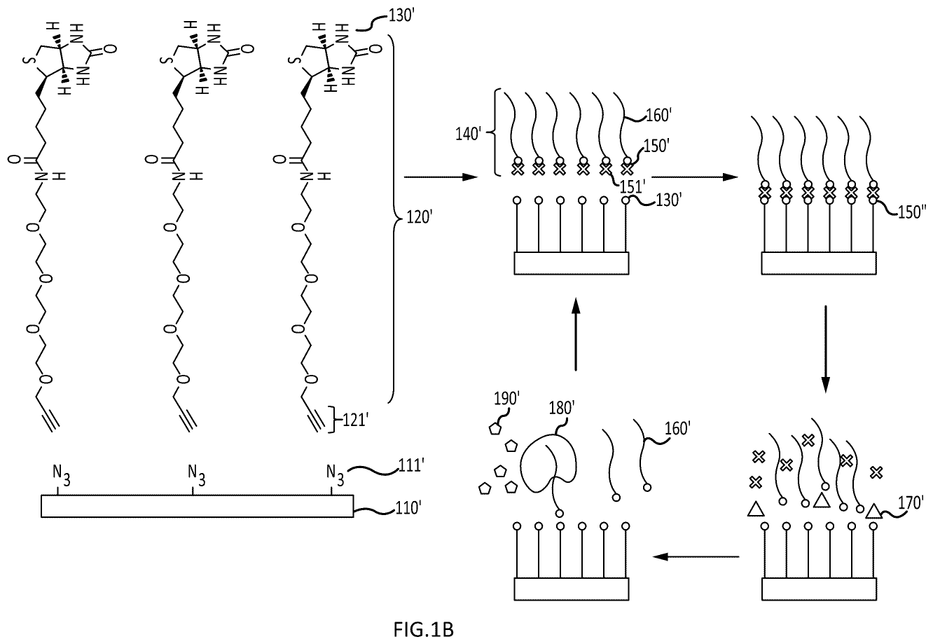

Illustratively, FIG. 1A illustrates a flow of example operations for using and reusing a flowcell, and example structures formed using such operations. The flow of example operations includes providing surface 110. Surface 110 may be in a flowcell. Surface 110 may include a substrate. For example, surface 110 may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof.

Molecules 120 are bound (e.g., covalently) to surface 110 in order to provide a moiety to which complexes may be reversibly coupled and decoupled in subsequent steps so as to provide flowcell reusability. In some examples, each molecule 120 includes moiety 121 and moiety 130. Moiety 121 may react with moiety 111, attached to surface 110, thereby binding molecule 120 to surface 110. Moiety 111 and moiety 121 may include any suitable pair of reactive moieties that form a linkage which is substantially irreversible during operations such as described with reference to FIG. 1A. For example, moiety 111 and moiety 121 may include an amine-NHS pair, an amine-imidoester pair, an amine-pentofluorophenyl ester pair, an amine-hydroxymethyl phosphine pair, an amine-carboxylic acid pair, a thiol-maleimide pair, a thiol-haloacetyl pair, a thiol-pyridyl disulfide pair, a thiol-thiosulfonate pair, a thiol-vinyl sulfone pair, an aldehyde-hydrazide pair, an aldehyde-alkoxyamine pair, a hydroxy-isocyanate pair, an azide-alkyne pair, an azide-phosphine pair, an azide-cyclooctyne pair, an azide-norbornene pair, a transcycloctene-tetrazine pair, a norbornene-tetrazine pair, an oxime, a SpyTag-SpyCatcher pair, a SNAP-tag-O⁶-benzylguanine pair, a CLIP-tag-O²-benzylcytosine pair, or a sortase coupling. Non-limiting examples of moiety pairs 111, 121 that may be used to couple molecule 120 to surface 110 are shown in Table 1 below.

TABLE 1

| Bonding pair | Example moiety 111 or 121 | Example moiety 121 or 111 |
|---|---|---|
| amine-NHS | amine group, —NH₂ | 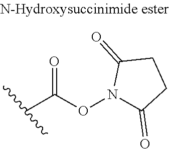 [SMILES: *C(=O)ON1C(=O)CCC1=O] |
| amine-imidoester | amine group, —NH₂ | 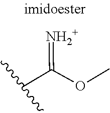 [SMILES: *C(=[NH2+])OC] |
| amine- pentofluorophenyl ester | amine group, —NH₂ | 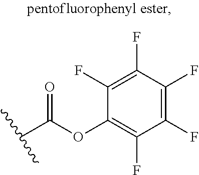 [SMILES: *C(=O)Oc1c(F)c(F)c(F)c(F)c1F] |
| amine- hydroxymethyl phosphine | amine group, —NH₂ | 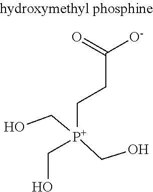 [SMILES: O=C([O-])CC[P+](CO)(CO)CO] |
| amine-carboxylic acid | amine group, —NH₂ | carboxylic acid group, —C(=O)OH (e.g., |
| | | following activation of the carboxylic acid by a |
| | | carbodiimide such as EDC (1-ethyl-3-(-3- |
| | | dimethylaminopropyl)carbodiimide |
| | | hydrochloride) or DCC (N′,N′-dicyclohexyl |
| | | carbodiimide) to allow for formation of an amide |
| | | bond of the activated carboxylic acid with an |
| | | amine group) |
| thiol-maleimide | thiol, —SH | 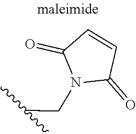 [SMILES: *CN1C(=O)C=CC1=O] |
| thiol-haloacetyl | thiol, —SH | 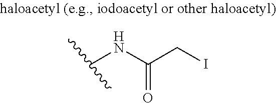 [SMILES: *NC(=O)CI] |
| thiol-pyridyl disulfide | thiol, —SH | 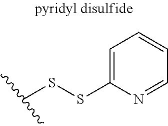 [SMILES: *SSc1ccccn1] |
| thiol-thiosulfonate | thiol, —SH | 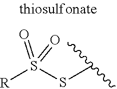 |
| thiol-vinyl sulfone | thiol, —SH | 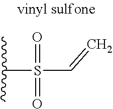 [SMILES: *S(=O)(=O)C=C] |
| aldehyde-hydrazide | aldehyde, —C(═O)H | 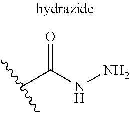 [SMILES: *C(=O)NN] |
| aldehyde-alkoxyamine | aldehyde, —C(═O)H | 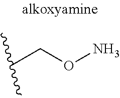 [SMILES: *CON] |
| hydroxy-isocyanate | hydroxyl, —OH | 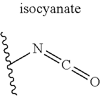 [SMILES: *N=C=O] |
| azide-alkyne | azide, —N₃ | 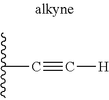 [SMILES: *C#C] |
| azide-phosphine | azide, —N₃ | 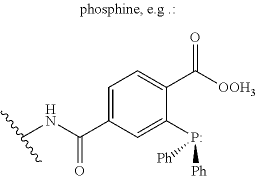 |
| azide-cyclooctyne | azide, —N₃ | 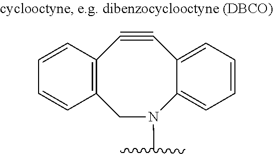 [SMILES: *N1Cc2ccccc2C#Cc2ccccc21] |
| | | 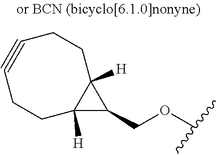 [SMILES: *OC[C@H]1[C@@H]2CCC#CCC[C@@H]21] |
| azide-norbornene | azine, —N₃ | 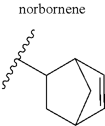 [SMILES: *C1CC2C=CC1C2] |
| transcyclooctene-tetrazine | 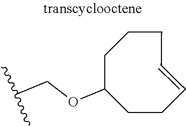 [SMILES: *COC1CC/C=C/CCC1] | 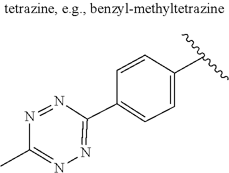 [SMILES: *c1ccc(-c2nnc(C)nn2)cc1] |
| norbornene-tetrazine | 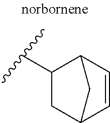 [SMILES: *C1CC2C=CC1C2] | 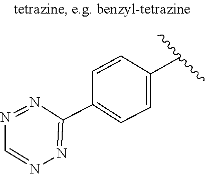 [SMILES: *c1ccc(-c2nncnn2)cc1] |
| oxime | aldehyde or ketone (e.g., amine | alkoxyamine |
| | cugroup or N-terminus of | |
| | polypeptide converted to an | |
| | aldehyde or ketone by pyroxidal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO: 1) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK (SEQ ID |
| | | NO: 2) |
| SNAP-tag-O⁶-Benzylguanine | SNAP-tag (O-6-methylguanine- DNA methyltransferase) | 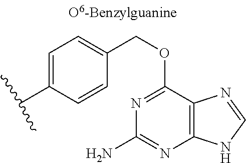 [SMILES: *c1ccc(COc2nc(N)nc3[nH]cnc23)cc1] |
| CLIP-tag-O²-benzylcytosine | CLIP-tag (modified O-6- methylguanine-DNA methyltransferase) | 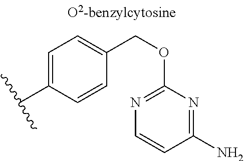 [SMILES: *c1ccc(COc2nccc(N)n2)cc1] |
| Sortase-coupling | -Leu-Pro-X-Thr-Gly (SEQ ID | -Gly₍₃₋₅₎ |
| | NO: 3) | |

As noted above, each molecule 120 includes a moiety 130. Moiety 130 may be coupled to moiety 121 of that molecule via a suitable element, such as a linker 122. Nonlimiting examples of linkers include an alkyl chain or a polymer. Nonlimiting examples of polymers for use in linker 122 include polyether, polyamide, polyester, polyaryl, poly(ethylene glycol) (PEG), or the like. Illustratively, linker 122 may include PEG having between 1 and 10 ethylene glycol units, e.g., PEG1 to PEG10, illustratively PEG2 to PEG6, such as PEG4.

Moiety 130 may be used to reversibly couple complex 140 to the surface 110, so as to reversibly couple an oligonucleotide to the surface. For example, complex 140 may include moiety 150 which couples to moiety 130, and at least one oligonucleotide 160 coupled to moiety 150. In some examples, complex 140 includes central molecule 151 (such as a protein), that includes one or more active sites to which other elements may be coupled. For example, oligonucleotide 160 may be coupled to a first active site of central molecule 151, e.g., via covalent or non-covalent interaction between a moiety coupled to the oligonucleotide and the first active site (in this, note that active sites may be considered moieties). In some examples, moiety 150 may correspond to a second active site on the central molecule 151.

Oligonucleotide 160 may be single-stranded, and optionally may be bound to moiety 150 via a suitable element, such as a linker (which may be configured similarly as linker 122). Oligonucleotide 160 may be or include a capture primer such as may be used for seeding and/or amplifying a template polynucleotide.

Complex 140 may be coupled to surface 110 via reaction between moiety 130 coupled to surface 110 and moiety 150 of complex 140. Moiety 130 and moiety 150 may include any suitable pair of reactive moieties that couple to one another in a way that is substantially irreversible during certain operations such as described with reference to FIG. 1A (e.g., during seeding, amplifying, and sequencing template polynucleotides), and subsequently may be decoupled from one another during one or more other operations such as described with reference to FIG. 1A (e.g., when regenerating the flowcell for reuse after sequencing). In some examples, moiety 150 may form a non-covalent bond with moiety 130, which may facilitate subsequent removal of complex 140 from the flowcell so that the flowcell may be reused in a manner such as described herein.

In some examples, moiety 130 may be selected so as to bond to an active site (moiety 150) of molecule 151. For example, moiety 130 and molecule 151 may include a biotin/streptavidin pair, a polyhistidine-tag (His-tag)/transition metal pair, a DIG/anti-DIG pair, a c-myc/anti-cmyc pair, a GST/glutathione pair, or a FLAG/anti-FLAG pair. Non-limiting examples of pairs of moiety 130 and central molecule 151 that may be used to couple molecule 120 to complex 140 are shown in Table 2 below. Additionally, non-limiting examples of reagents that may be used to decouple molecule 120 from complex 140 are shown in Table 2.

TABLE 2

| | | | Example reagent(s) for |
|---|---|---|---|
| | | | decoupling moiety 130 |
| | Example moiety | Example central | from central molecule |
| Bonding pair | 130 | molecule 151 | 151 |
| biotin-streptavidin | biotin, | Streptavidin, | formamide or ethylene |
| | desthiobiotin, dual- | neutravidin, avidin, | glycol |
| | biotin, Strep-tag | Strep-Tactin | |
| His-tag transition | transition metal | Histidine tag (His-tag) | imidazole |
| metal | (e.g., Mn²⁺, Fe²⁺, | | |
| | Co₂₊, Ni²⁺, or Cu²⁺) | | |
| His-tag transition | His-tag | transition metal (e.g., | imidazole |
| metal | | Mn²⁺, Fe²⁺, Co₂₊, Ni²⁺, | |
| | | or Cu²⁺) | |
| DIG/anti-DIG | digoxigenin (DIG) | anti-digoxigenin (anti- | Glycine approx. pH 3.0 |
| | | DIG) antibody | or approx. 3M sodium |
| | | | thiocyanate (NaSCN) |
| c-myc/anti-cmyc | c-myc (also referred | anti-cmyc antibody | Glycine approx. pH 3.0 |
| | to as MYC) | | or approx. 3M sodium |
| | | | thiocyanate (NaSCN), or |
| | | | competition with c-myc |
| | | | peptide |
| c-myc/anti-cmyc | anti-cmyc antibody | c-myc | Competition with c-myc |
| | | | peptide |
| GST/glutathione | glutathione | glutathione s-transferase | Competition with excess |
| | | (GST) | glutathione |
| GST/glutathione | glutathione s- | glutathione | Competition with excess |
| | transferase (GST) | | glutathione |
| FLAG/anti-FLAG | FLAG tag | Anti-FLAG antibody | Glycine approx. pH 3.5 |
| | | | or competition with |
| | | | excess FLAG peptide |
| FLAG/anti-FLAG | Anti-FLAG | FLAG tag | Competition with excess |
| | antibody | | FLAG peptide |

In one nonlimiting example, molecule 151 is or includes a protein having multiple active sites (moieties 150). Oligonucleotide 160 may be coupled to a moiety that is coupled to a first one of the active sites (e.g., via its own moiety 130 such as exemplified in Table 2), and moiety 130 of molecule 120 may be coupled to a second one of the active sites. Illustratively, molecule 151 may be or include streptavidin or related protein (e.g., neutravidin, avidin, or Strep-Tactin); oligonucleotide 160 may be coupled to biotin or related moiety (e.g., desthiobiotin, dual-biotin, or Strep-tag) that is coupled to a first active site of the molecule 151; and moiety 130 may be or include another biotin or related moiety (e.g., desthiobiotin, dual-biotin, or Strep-tag) that is coupled to a second active site of the molecule 151. Optionally, complex 140 may include multiple oligonucleotides 160, e.g., coupled to different active sites of molecule 151. The oligonucleotides may have the same sequences as one another (e.g., may all be P5, or may all be P7). Alternatively, the oligonucleotides may have different sequences than one another (e.g., may be a mixture of P5 and P7). In nonlimiting examples where molecule 151 includes streptavidin or related protein, the protein may be incubated ahead of time with multiple P5 and/or P7 oligonucleotides 160 which are coupled to respective biotins or related moieties to form a complex which may referred to herein as “streptavidin-dualbiotin-P5/P7”. In some examples, the resulting complex 140 may include one, two, or three oligonucleotides 160, leaving at least one available active site available to bind with biotin or related moiety at surface 110 in a manner such as illustrated in FIG. 1A. As another example, molecule 151 may be or include His-tag which is coupled directly or indirectly to oligonucleotide 160, moiety 130 may be or include a transition metal that is coupled to an active site of the His-tag. As another example, molecule 151 may be or include a transition metal which is coupled directly or indirectly to oligonucleotide 160, moiety 130 may be or include a His-tag with an active site that is coupled to the transition metal.

Oligonucleotides 160 may be used for seeding, clustering, and sequencing processes (not specifically illustrated), e.g., may be used as primers to generate clusters of amplicons that may be sequenced using sequencing-by-synthesis. Following such sequencing, the flowcell may be regenerated and then reused. For example, reagent 170 may be introduced to decouple complex 140 from moiety 130. For example, reagent 170 may interfere with the bond between moiety 150 and moiety 130 (illustratively, by denaturing central molecule 151, or by competing with the bond between moiety 150 and moiety 130) thereby decoupling complex 140 from moiety 130.

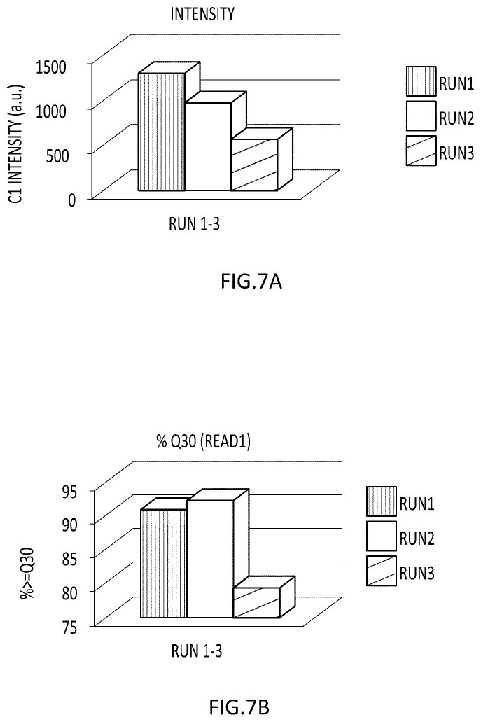

A nuclease digest is also performed such that nuclease 180 digests polynucleotides in the flowcell (e.g., oligonucleotides 160, and any polynucleotides coupled thereto), into nucleotides 190. Nuclease 180 may include any appropriate nuclease. For example, nuclease 180 may include a polymerase, illustratively a DNA polymerase that has 3′ to 5′ exonuclease activity. Additionally, or alternatively, nuclease 180 may include an exonuclease (such as Exonuclease I, also referred to as ExoI). Additionally, or alternatively, nuclease 180 may include a non-specific dsDNA nuclease (such as DNaseI). Additionally, or alternatively, nuclease 180 may include Micrococcal Nuclease (MNase). MNase digests 5′-phosphodiester bonds of DNA and RNA, yielding 3′-phosphate mononucleotides and oligonucleotides. For further details regarding MNase, see the following references, the entire contents of which are incorporated by reference herein: Cuatrecasas et al., “Catalytic properties and specificity of the extracellular nuclease of Staphylococcus aureus,” J. Biol. Chem. 242(7): 1541-1547 (1967); Craig et al., “Plasmid cDNA-directed protein synthesis in a coupled eukaryotic in vitro transcription-translation system,” Nucleic Acids Res. 20(19): 4987-4985 (1992); and O'Neill et al., “Immunoprecipitation of native chromatin: NChIP,” Methods 31(1): 86-82 (2003). In some examples, nuclease 180 may include a combination of different nucleases, such as a combination of DNaseI and ExoI. In other examples, nuclease 180 may consist essentially of MNase. Illustratively, the inventors have observed that MNase is particularly potent, giving significantly less contamination from run to run when compared to a combination of DNaseI and ExoI (so the single enzyme MNase may in some examples perform better than the 2 enzymes together). Further details are provided with reference to FIG. 7 below.

At any suitable time after using reagent 170 and nuclease 180, the surface 110 may be washed, a new set of molecules 140 can be introduced, and the cycle repeated. The new set of complexes 140 may be of the same type or of a different type than the original set of complexes 140. Illustratively, the new set of complexes may include oligonucleotides having the same sequence as oligonucleotides 160, or having one or more different sequences than oligonucleotides 160.

FIG. 1B illustrates a nonlimiting example of elements that may be used in the example of FIG. 1A. Referring now to FIG. 1B, in a non-limiting example, surface 110′ includes poly(N-(5-azideoacetamidylpentyl)acrylamide-co-acrylamide) (PAZAM). PAZAM surface 110′ includes azide moieties 111′ which may be reacted with moieties 121′ in molecules 120′ to couple the molecules to the surface. For example, molecules 120′ may include alkynes (such as alkyne, BCN, or DBCO) as moieties 121, PEG1 to PEG10 as linker 122, and biotin as moiety 130. In the nonlimiting example in which PEG4 is the linker, molecules 120′ may be referred to as alkyne-PEG4-biotin molecules 120′. Once attached to surface 110′ via reaction between moieties 111′ and 121′, biotin 130′ is available to be reacted. In the nonlimiting example shown in FIG. 1B, biotin 130′ is then reacted with an active site 150″ of a pre-incubated streptavidin-dualbiotin-P5/P7 complex 140′, binding complexes 140′ to surface 110′. Each streptavidin-dualbiotin-P5/P7 complex 140′ include a streptavidin central molecule 151′ and at least one oligonucleotide 160′, e.g., one or more P5 and/or P7 oligonucleotides which are functionalized to dualbiotin to which a respective active site (moiety 150′) of streptavidin binds. At this point, oligonucleotides 160′ can be used for appropriate clustering and sequencing processes (not specifically illustrated). To initiate regeneration and reuse of the flowcell, a reagent such as hot formamide or ethylene glycol 170′ is introduced to decouple streptavidin central molecule 151′ from biotin 130′, for example by denaturing the streptavidin. A nuclease digest is also performed such that nuclease 180′ digests polynucleotides in the flowcell, e.g., oligonucleotides 160′ and any polynucleotides coupled thereto, into nucleotides 190′. At this point, or at a later time, another set of complexes 140′ can be introduced and the cycle repeated.

Additionally, kits are provided herein that include one or more elements such as described with reference to FIG. 1A or FIG. 1B. For example, a kit may include a plurality of molecules 120 configured to be coupled to surface 110. Each molecule 120 includes first moiety 130. The kit may include a plurality of complexes 140. Each complex 140 includes second moiety 150, which can couple to first moiety 130. Each complex 140 also includes oligonucleotide 160 which is coupled to second moiety 150. The kit may include a reagent 170 to decouple first moiety 130 and second moiety 150 from one another. The kit may include at least one nuclease 180 to digest polynucleotides. Note, however, that kits need not necessarily include molecules 120. Instead, the flowcell may come with a surface that already includes first moiety 130 which is ready to be coupled to complexes 140 in the kit.

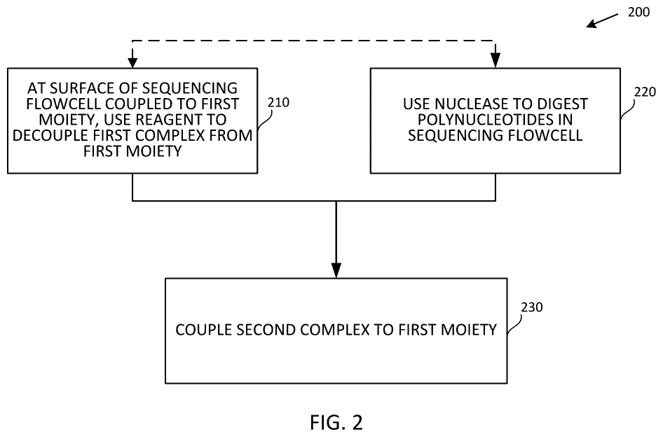

FIG. 2 illustrates a flow chart showing the flow of operations in an example automated method 200 conducted in a sequencing flowcell. Method 200 may include, at the surface of the sequencing flowcell coupled to a first moiety, using a reagent to decouple a first complex from the first moiety (operation 210). Nonlimiting examples of the reagent used at operation 210 are provided in Table 2 for different central molecules 151 and different moieties 130 that may be coupled to the flowcell. The first moiety referred to in operation 210 may correspond to moiety 130 described with reference to FIG. 1A, illustratively moiety 130′ described with reference to FIG. 1B. The first complex may correspond to complex 140 described with reference to FIG. 1A, illustratively complex 140′ described with reference to FIG. 1B. The first complex may include a second moiety which couples to the first moiety. In some examples, the second moiety may couple to the first moiety via a non-covalent bond. The second moiety may correspond to moiety 150 as described with reference to FIG. 1A, illustratively moiety 150″ described with reference to FIG. 1B. The regent may work, for example, by denaturing at least part of complex 140 (illustratively, by denaturing central molecule 151, such as in the example described with reference to FIG. 1B), or by competing with moiety 130 to bond to moiety 150.

The first complex referred to in operation 210 may also include a polynucleotide coupled to the second moiety. The polynucleotide may include, or may correspond to, oligonucleotide 160 as described with reference to FIG. 1A, illustratively oligonucleotide 160′ described with reference to FIG. 1B. The polynucleotide may have been used for appropriate clustering and sequencing processes, and as such may include oligonucleotide 160 or 160′ which is extended to include an amplicon of a template polynucleotide. Method 200 may also include using a nuclease to digest polynucleotides in the sequencing flowcell (operation 220). For example, the nuclease may digest the polynucleotide coupled to the second moiety or a different polynucleotide in the flow cell. It should be noted that operation 210 may be performed either before or after operation 220, or even at the same time as operation 220, as intended to be indicated by the dashed arrow. Wash steps may be performed before and/or after each of operations 210 and 220. For example, operation 210 may be performed, the surface washed to remove decoupled complexes, operation 220 then performed to digest any remaining polynucleotides, and the surface washed again to remove digested polynucleotides. Or, for example, operation 220 may be performed to digest polynucleotides, the surface washed to remove digested polynucleotides, operation 210 may be performed to decouple complexes (from which the polynucleotides may have been at least partially removed), and the surface washed again to remove the decoupled complexes.

Method 200 may also include, after using the reagent and after using the nuclease, coupling a second complex to the first moiety (operation 230). The second complex 140 or 140′ may include a third moiety 150 or 150′ which couples with the first moiety 130 or 130′ and an oligonucleotide 160 or 160′ coupled to the third moiety. In some examples, the third moiety 150 or 150′ may couple to the first moiety 130 or 130′ via a non-covalent bond. Nonlimiting examples of moieties 130, 130′, 150, and 150′ are provided elsewhere herein. In some examples, the second moiety (e.g., 150 or 150′ of the first complex) is coupled to the first moiety 130 or 130′ via a first non-covalent bond, and the third moiety (e.g., 150 or 150′ of the second complex) is coupled to the first moiety 130 or 130′ via a second non-covalent bond).

In some examples, the surface may be coupled to the first moiety by reaction of a fourth moiety at the surface with a fifth moiety of a molecule including the first moiety. The fourth moiety may correspond to moiety 111 as described with reference to FIG. 1A, illustratively moiety 111′ described with reference to FIG. 1B. The fifth moiety may correspond to moiety 121 as described with reference to FIG. 1A, illustratively moiety 121′ described with reference to FIG. 1B. Nonlimiting examples of moieties 111, 111′, 121, and 121′ are provided elsewhere herein, e.g., with reference to Table 1. Illustratively, the fourth moiety may include azide, and the fifth moiety may include an alkyne (e.g., alkyne, BCN, or DBCO). Optionally, the molecule may further include a linker disposed between the fifth moiety and the first moiety. In some examples, the linker may include poly(ethylene glycol), e.g., PEG1 to PEG10 (illustratively, PEG4).

In some examples, method 200 may further include, before using the reagent and before using the nuclease, sequencing the polynucleotide. For example, in a manner such as described with reference to FIGS. 1A and 1B, oligonucleotide 160 or 160′ may be used for seeding and/or amplifying a template polynucleotide, illustratively using sequencing-by-synthesis. Illustratively, method 200 may further include, after coupling the second complex to the first moiety, using the oligonucleotide to amplify a template polynucleotide, and sequencing the amplified template polynucleotide. The flowcell may be regenerated and reused at any suitable time after the sequencing, and optionally stored at any suitable time. The regenerated flowcell may be stored in a suitably controlled environment to inhibit degradation of the moieties which are coupled to the surface at the time of storage.

For example, the flowcell may be washed and optionally stored after the second complex is coupled to the first moiety, and before the oligonucleotide is used to amplify the template polynucleotide. In this case, the flowcell may be ready for the next sequencing run as soon as the previous run finishes, and can be stored in this ready state. Alternatively, the flowcell may be washed and optionally stored after the polynucleotide is sequenced, before using the reagent, before using the nuclease, and before the second complex is coupled to the first moiety. Performing decoupling, digesting, and rebinding steps at the beginning of the sequencing run may delay the start of clustering, but may reduce the amount of time for which reagent 170 may be exposed to air which otherwise may cause degradation of the reagent. As yet another alternative, the flowcell may be washed and optionally stored after the polynucleotide is sequenced, after using the reagent, after using the nuclease, and before the second complex is coupled to the first moiety. In this example, the decoupling and digesting are performed at the end of the sequencing run, leaving the flowcell surface clean. The flowcell then can be regenerated by coupling a fresh set of complexes to the surface at the beginning of the sequencing run. Such re-binding is expected to cause only a short delay at the beginning of the sequencing run.

Note that all operations besides storing the flowcell (which entails removing the flowcell from the sequencer and moving it to a storage area) may be performed automatically by the sequencer. For example, the sequencer may be configured to receive a kit such as described above, e.g., that includes the reagent, nuclease, and complexes, and may be configured to automatically flow such elements into the flowcell at appropriate times to couple the complexes to the flowcell surface before sequencing, to use the reagent to decouple the complexes from the flowcell surface after sequencing, and to use the nuclease to digest any remaining polynucleotides within the flowcell before coupling additional complexes to the surface.

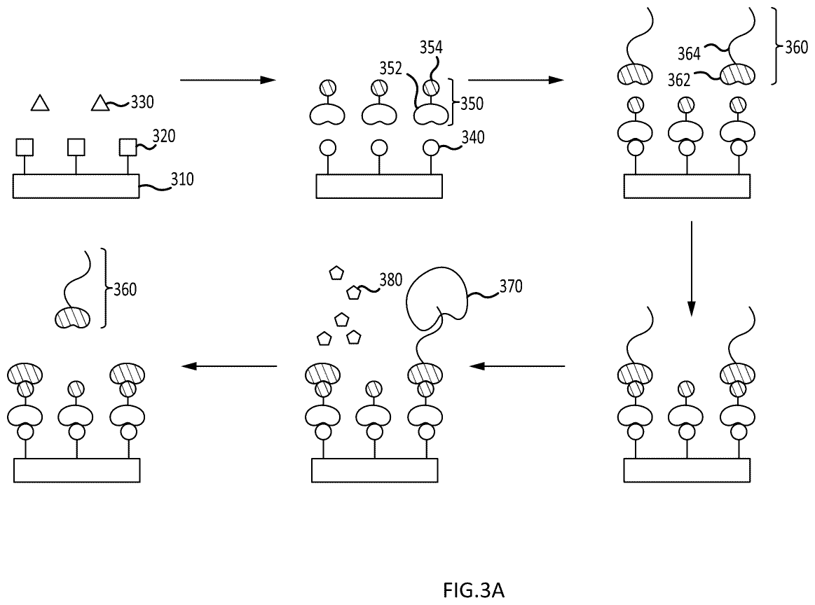

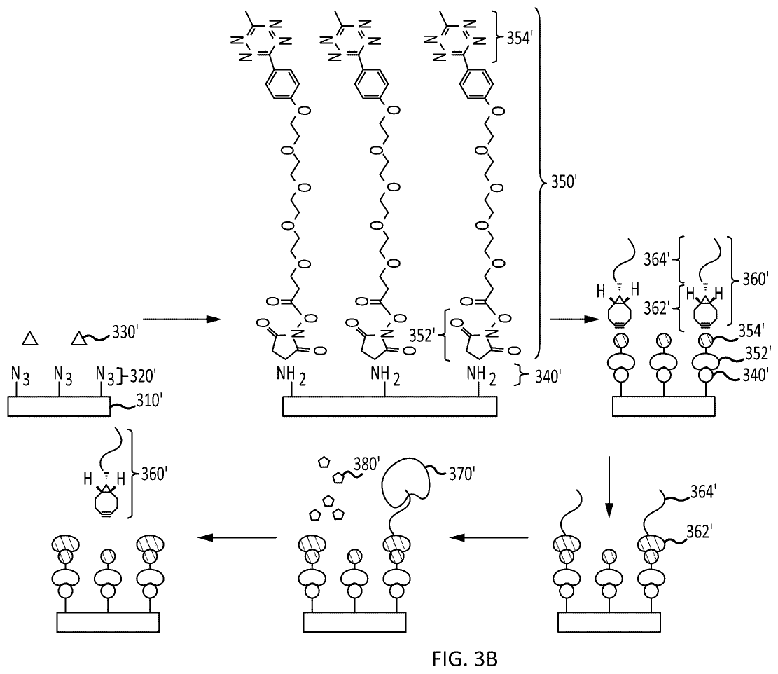

It will be appreciated that complexes such as described with reference to FIGS. 1A-1B and 2 provide just one example of elements that may be used to reuse a flowcell. For example, FIG. 3A illustrates a flow of example operations for using and reusing a flowcell, and example structures formed using such operations. The flow of example operations includes providing surface 310. Surface 310 may be in a flowcell. Nonlimiting examples of substrates that may be used in a flowcell are provided elsewhere herein, e.g., with reference to FIGS. 1A-2B. As illustrated in FIG. 3A, surface 310 includes moieties 320. Moieties 320 may be the natural termination of surface 310. Illustratively, in examples in which surface 310 includes PAZAM, moieties 320 may be azide. Moieties 320 may be converted into moieties 340 using reagent 330. Reagent 330 may be any suitable reagent, for example, a reducing agent. Non-limiting examples of reducing agents include tris(hydroxypropyl)phosphine (THP), lithium aluminum hydride (LiAlH₄), Red-Al (NaAlH₂(OCH₂CH₂OCH₃)₂), hydrogen (H₂), sodium amalgam (Na(Hg)), sodium-lead alloy, zinc amalgam (Zn(Hg)), diborane, sodium borohydride (NaBH₄), a ferrous compound, a stannous compound, sulfur dioxide, a diothionate, a thiosulfate, an iodide, hydrogen peroxide, hydrazine, diisobutylaluminum hydride (DIBAL-H), oxalic acid (C₂H₂O₄), formic acid (HCOOH), ascorbic acid (C₆H₈O₆), a reducing sugar, a phosphite, a hypophosphite, phosphorous acid, dithiothreitol (DTT), carbon monoxide (CO), a cyanide, carbon, tris-2-carboxyethylphosphine hydrochloride (TCEP), or any suitable combination thereof. Moiety 340 may be the reduced form of moiety 320.

Molecules 350 are bound (e.g., covalently) to surface 310 in order to provide a moiety to which oligonucleotides may be coupled and decoupled in subsequent steps so as to provide flowcell reusability. In some examples, each molecule 350 includes moiety 352 and moiety 354. Moiety 352 may react with moiety 340, attached to surface 310, thereby binding molecule 350 to surface 110. Moiety 340 and moiety 340 may include any suitable pair of reactive moieties that form a linkage which is substantially irreversible during operations such as described with reference to FIG. 3A. For example, moiety 340 and moiety 352 may include an amine-NHS pair, an amine-imidoester pair, an amine-pentofluorophenyl ester pair, an amine-hydroxymethyl phosphine pair, an amine-carboxylic acid pair, a thiol-maleimide pair, a thiol-haloacetyl pair, a thiol-pyridyl disulfide pair, a thiol-thiosulfonate pair, a thiol-vinyl sulfone pair, an aldehyde-hydrazide pair, an aldehyde-alkoxyamine pair, a hydroxy-isocyanate pair, an azide-alkyne pair, an azide-phosphine pair, an azide-cyclooctyne pair, an azide-norbornene pair, a transcycloctene-tetrazine pair, a norbornene-tetrazine pair, an oxime, a SpyTag-SpyCatcher pair, a SNAP-tag-O⁶-benzylguanine pair, a CLIP-tag-O²-benzylcytosine pair, or a sortase coupling. Non-limiting examples of moiety pairs 340, 352 that may be used to couple molecule 350 to surface 310 may be the same as those provided in Table 1 further above for moiety pairs 111, 121.

In a manner similar to that noted above for molecule 120, each molecule 350 optionally includes a linker or other suitable element (not specifically labeled) coupling moiety 354 to moiety 352. Nonlimiting examples of linkers include those described with reference to linker 122 illustrated in FIG. 1A.

Molecules 350 may be used to couple to surface 310 additional molecules 360 which include respective oligonucleotides 364. For example, moiety 354 is able to react with moiety 362 to bind molecule 360 to the surface 310. Moiety 354 and moiety 362 may include any suitable pair of reactive moieties. For example, moiety 354 and moiety 362 may include an amine-NHS pair, an amine-imidoester pair, an amine-pentofluorophenyl ester pair, an amine-hydroxymethyl phosphine pair, an amine-carboxylic acid pair, a thiol-maleimide pair, a thiol-haloacetyl pair, a thiol-pyridyl disulfide pair, a thiol-thiosulfonate pair, a thiol-vinyl sulfone pair, an aldehyde-hydrazide pair, an aldehyde-alkoxyamine pair, a hydroxy-isocyanate pair, an azide-alkyne pair, an azide-phosphine pair, an azide-cyclooctyne pair, an azide-norbornene pair, a transcycloctene-tetrazine pair, a norbornene-tetrazine pair, an oxime, a SpyTag-SpyCatcher pair, a SNAP-tag-O⁶-benzylguanine pair, a CLIP-tag-O²-benzylcytosine pair, or a sortase coupling. Non-limiting examples of moiety pairs 340, 352 that may be used to couple molecule 350 to surface 310 may be the same as those provided in Table 1 further above for moiety pairs 111, 121.

In a manner similar to that described with reference to FIG. 1A, oligonucleotide 364 may be, or include, a primer, and may be used in clustering and sequencing processes. Note that a portion of moieties 354 remain unreacted, and these unreacted moieties may be used subsequently to couple fresh oligonucleotides 364 to the flowcell in order to reuse the flowcell. More specifically, to reuse the flowcell, a nuclease digest is performed in a manner similar to that described with reference to FIG. 1A, such that nuclease 370 digests polynucleotides in the flowcell, e.g., oligonucleotides 364 coupled to molecules 360, into nucleotides 390. Nuclease 370 may include any appropriate nuclease. Nonlimiting examples of nucleases are provided elsewhere herein. In one nonlimiting example, nuclease 370 may include MNase. In some examples, nuclease 370 may include a combination of different nucleases, such as a combination of DNaseI and ExoI. In other examples, nuclease 370 may consist essentially of MNase. Illustratively, the inventors have observed that MNase is particularly potent, giving significantly less contamination from run to run when compared to a combination of DNaseI and ExoI (so the single enzyme MNase is in some examples better than the 2 enzymes). Further details are provided with reference to FIG. 7 below.

At this point, or at a later time, another set of molecules 360 can be introduced and reacted with previously unreacted moieties 354. The new set of molecules 360 may be of the same type or a different type than the original set of molecules 360.

Referring now to FIG. 3B, in a non-limiting example, surface 310′ includes PAZAM including azide moieties 320′. The azide moieties 320′ are reacted with reducing agent 330′ to convert them into amine moieties 340′. Amine moieties 340′ then are reacted with molecules 350′. In the illustrated example, molecules 350′ are methyltetrazine-PEG4-NHS, in which NHS moiety 352′ reacts with amine moieties 340′, PEG4 is a linker, and tetrazine moiety 354′ available to react with molecules 360′. Molecules 360′ include alkyne moieties, such as BCN 362′, which is coupled to an oligonucleotide 364′ such as a P5 or P7 primer (in this nonlimiting example, molecules 360′ may be referred to as BCN-P5/P7 molecules). A portion of tetrazine moieties 354′ react with BCN moieties 362′, attaching BCN-P5/P7 molecules 360′ (including oligonucleotide 364′) to surface 310′. A portion of tetrazine moieties 354′ remain unreacted. At this point, oligonucleotides 364′ can be used for appropriate clustering and sequencing processes. To enable reusability of the flowcell, a nuclease digest may be performed such that MNase 370′ digests polynucleotides 375′ into nucleotides 390′. At this point, another set of molecules 360′ (e.g., BCN-P5/P7 molecules) can be introduced and reacted with previously unreacted moieties 354′.

Additionally, kits are provided herein that include one or more elements such as described with reference to FIG. 3A. For example, a kit may include surface 310 including first moieties 320. The kit may also include first molecules 330 to convert first moieties 320 to second moieties 340. The kit may also include second molecules 350 each including third moiety 352 and fourth moiety 354. Third moiety 352 may be able to react with one of second moieties 340 to couple second molecule 350 to surface 310. The kit may also include third molecules 360. Each third molecule 360 may include fifth moiety 362 and oligonucleotide 364 coupled to fifth moiety 362. Fifth moiety 362 may be able to react with one of fourth moieties 354 to couple oligonucleotide 364 to surface 310. The kit may also include nuclease 370 configured to digest polynucleotides 375 that are sequenced in the flowcell using oligonucleotides 364 of molecules 360. Nuclease 370 may include, or may consist essentially of, MNase. Note, however, that kits need not necessarily include molecules 330 and/or molecules 350, and instead may come with flowcell surfaces that already include fourth moiety 354 which is ready to be coupled to molecules 360 in the kit.

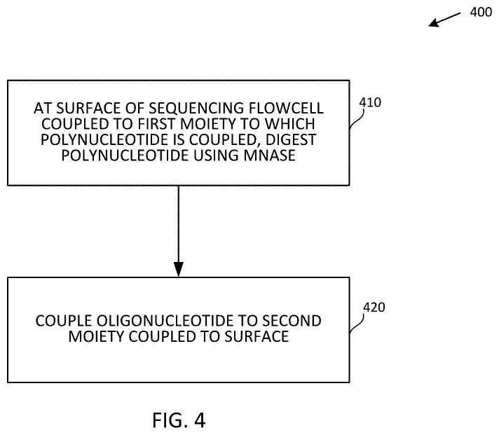

FIG. 4 illustrates a flow chart showing the flow of operations in another example automated method 400 conducted in a sequencing flowcell. Method 400 may include, at a surface of the sequencing flowcell coupled to a first moiety to which a polynucleotide is coupled, digesting the polynucleotide using MNase (operation 410). The first moiety referred to in operation 410 may correspond to moiety 362 as described with reference to FIG. 3A, illustratively moiety 362′ described with reference to FIG. 3B. The polynucleotide may correspond to polynucleotide 364 as described with reference to FIG. 3A, illustratively polynucleotide 364′ described with reference to FIG. 3B. The MNase may correspond to nuclease 370 as described with reference to FIG. 3A, and MNase 370′ described with reference to FIG. 3B.

Method 400 may also include, after digesting the polynucleotide, coupling an oligonucleotide to a second moiety coupled to the surface (operation 420). The second moiety may correspond to an unreacted moiety 354, as described with reference to FIG. 3A. In a nonlimiting example, unreacted moiety 354 may include a tetrazine moiety, as described with reference to FIG. 3B. In some examples, coupling the oligonucleotide to the second moiety includes reacting the second moiety with a third moiety of a molecule that comprises the oligonucleotide. The molecule comprising the oligonucleotide may correspond to molecule 360 described with reference to FIG. 3A, illustratively molecule 360′ described with reference to FIG. 3B. As such, third moiety may correspond to moiety 362 as described with reference to FIG. 3A, illustratively moiety 362′ described with reference to FIG. 3B. Moiety 354 or 354′ and moiety 362 or 362′ may include any suitable pair of reactive moieties, some nonlimiting examples of which are provided elsewhere herein, e.g., in Table 1.

In some examples, the first moiety and the second moiety may be of the same type as one another. In other examples, the first moiety and the second moiety may be of different types than each other.

In some examples, the surface may be coupled to the first moiety via reaction of a fourth moiety at the surface with fifth moiety of a molecule including the first moiety. In some examples, the fourth moiety corresponds to moiety 340 as described with reference to FIG. 3A, illustratively moiety 340′ described with reference to FIG. 3B. In some examples, the fifth moiety corresponds to moiety 352 as described with reference to FIG. 3A, illustratively moiety 352′ described with reference to FIG. 3B. Moiety 340 and moiety 352 may include any suitable pair of reactive moieties, nonlimiting examples of which are provided elsewhere herein, e.g., in Table 1. In some examples, the molecule may further include a linker disposed between the fifth moiety and the first moiety.

In some examples, method 400 may further include, before using the MNase, sequencing the polynucleotide, e.g., in a manner such as described elsewhere herein. Illustratively, method 400 may include, after coupling the oligonucleotide to the second moiety, using the oligonucleotide to amplify a template polynucleotide, and sequencing the amplified template polynucleotide. The oligonucleotide may correspond to oligonucleotide 364 as described with reference to FIG. 3A, illustratively oligonucleotide 364′ as described with reference to FIG. 3B. The flowcell may be regenerated and reused at any suitable time after the sequencing, and optionally stored at any suitable time. The regenerated flowcell may be stored in a suitably controlled environment to inhibit degradation of the moieties which are coupled to the surface at the time of storage. For example, in a manner similar to that described elsewhere herein, the flowcell is washed and optionally stored after the oligonucleotide is coupled to the second moiety, and before the oligonucleotide is used to amplify the template polynucleotide. In other examples, the flowcell is washed and optionally stored after the polynucleotide is sequenced, before using the MNase, and before the oligonucleotide is coupled to the second moiety.

Similarly as described for method 200, note that all operations in method 400 besides optionally storing the flowcell (which entails removing the flowcell from the sequencer and moving it to a storage area) may be performed automatically by the sequencer. For example, the sequencer may be configured to receive a kit such as described above, e.g., that includes the nuclease and molecules to which oligonucleotides are coupled, and may be configured to automatically flow such elements into the flowcell at appropriate times to couple the molecules to the flowcell surface before sequencing, and to use the nuclease to digest any remaining polynucleotides within the flowcell before coupling additional molecules to the flowcell surface.

### WORKING EXAMPLES

The following examples are intended to be purely illustrative, and not limiting of the present invention.

### Example 1

A flowcell was prepared and used in the manner described with reference to FIGS. 1A-1B and FIG. 2. In this example, the flowcell used the reversible linkage between biotin and streptavidin. More specifically, a reversible, biotinylated surface was generated by grafting alkyne-PEG-biotin to the azides of a hydrogel coating. Next, a pre-incubated streptavidin-dualbiotin-P5/P7 complex was used to bind oligonucleotide primers to the surface, for use in clustering and sequencing template polynucleotides. To regenerate and reuse the surface, the biotin-streptavidin interactions were decoupled using hot formamide which denatured the streptavidin. A nuclease digest was used to inhibit oligonucleotides or polynucleotides from one run from being carried over into a subsequent run. The flowcell was then washed.

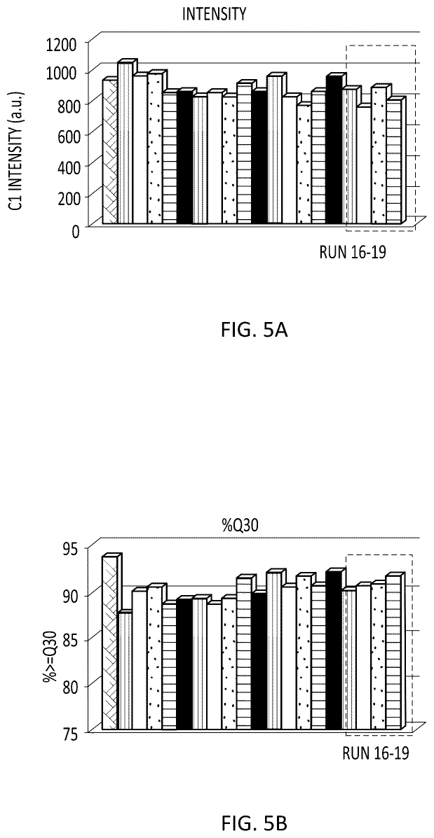

FIG. 5 illustrates data collected from surfaces prepared using this example. The data illustrated are collected from 19 high quality runs on a single flowcell using a NovaSeq instrument (Illumina, Inc.; San Diego, US). For the first 15 runs, the regeneration steps (e.g., binding with pre-incubated complexes, biotin-streptavidin dissociation, nuclease digest) were performed offline. For runs 16-19, the regeneration steps were performed using the NovaSeq, just before the next run. Runs 1-8 used DNaseI as the nuclease, and runs 9-19 used MNase as the nuclease. Instrument washing steps were performed separately. The flowcell was stored off-instrument in a hybridization buffer (HT1, 5×SSC with 0.1% Tween20) at a temperature of 4° C. between runs. As shown in plot A of FIG. 5, the cycle 1 (C1) intensity (referring to the brightness of the clusters as imaged at cycle 1 of sequencing) was substantially within the same range across all 19 runs. As shown in plot B of FIG. 5, the data quality was substantially preserved between all 19 runs. This indicates that the flowcell could satisfactorily be reused numerous times (here, at least 19 times) with no degradation in performance, e.g., with substantially no degradation in sequencing intensity and in sequencing quality. Additionally, substantially no difference in sequencing quality and intensity was observed between runs that were performed online and those that were performed offline, indicating that the flowcells could be regenerated at any suitable time relative to when the sequencing was performed, and that the flowcells satisfactorily may be regenerated either on instrument or manually. Of course, on-instrument regeneration of flowcells is particularly convenient and significantly reduces the burden on the end-user.

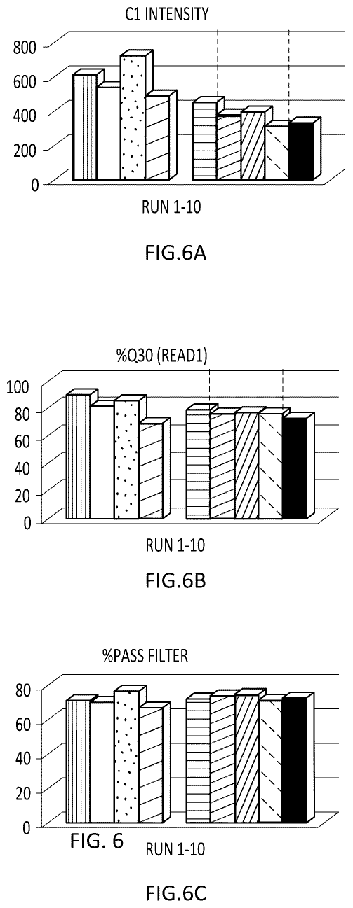

FIG. 6 illustrates data collected from 10 runs on a single flowcell using a NovaSeq instrument. In runs 1-6, the flowcell was regenerated off board, in runs 7-9 the flowcell was regenerated using the NovaSeq, and prior to run 10 a post-run wash was implemented. Run 5 failed, so the data for that run is omitted. Plot (A) of FIG. 6 shows that the C1 intensity was satisfactory through all 10 runs. Plot (B) of FIG. 6 shows that the data quality (% Q30) was satisfactory through all 10 runs. Indeed, the data quality was even more consistent when the flowcell was regenerated on-board as compared to when it was regenerated off board. Plot (C) of FIG. 7 shows that cluster monoclonality (% Pass Filter) remained substantially constant, and satisfactory, throughout the runs.

As such, it is expected that flowcells prepared, used, and reused in accordance with FIGS. 1A-1B and 2 may be reused for at least 5 times, or at least 10 times, or at least 15 times, or at least 20 times, or greater than 20 times, substantially without degradation in sequencing intensity, sequencing quality, and/or cluster monoclonality.

FIG. 7 illustrates the reduction of run-to-run contamination using a nuclease and additionally the improvement with MNase over DNaseI in the operations of Example 1. Data illustrated here was collected using a HiSeqX instrument sequencing a human DNA library followed by a second run sequencing a PhiX library after regenerating the flowcell either without a nuclease step or with a nuclease as will be described. The percentage alignment human reads within PhiX reads in the second run was used to determine how much DNA contamination remained on the flowcell between the first and second runs. In a first modification of operation 220 described with reference to FIG. 2, “no DNase” as shown in FIG. 7, the flowcell was reused without using a nuclease to digest polynucleotides in the flowcell before the next sequencing run. In the second modification of operation 220, “DNase I” as shown in FIG. 7, the flowcell was reused using a combination of DNaseI and ExoI to digest polynucleotides in the flowcell before the next sequencing run. In a third modification of operation 220, “MNase” as shown in FIG. 7, the flowcell was reused using only MNase to digest polynucleotides in the flowcell before the next sequencing run. A background run was performed without flowcell reuse as comparison.

It may be seen that reusing the flowcell without using a nuclease to digest polynucleotides between runs resulted in a percent alignment of about 3.4%, which was an unacceptably high level of contamination in the second run. In comparison, reusing the flowcell using a combination of DNAseI and ExoI to digest the previous runs' nucleotides resulted in a significantly lower level of contamination in the second run, about 0.012%. From this, it may be understood that using a nuclease between sequencing runs may reduce or inhibit contamination from the previous sequencing run. In still further comparison, reusing the flowcell using only MNase to digest the previous runs' nucleotides resulted in an even lower level of contamination in the second run, about 0.0019%. From this, it may be understood that using MNase may significantly reduce contamination from run to run. For example, MNase was observed here to provide about six times less contamination than the combination of DNaseI and ExoI. As such, the present inventors believe that MNase may be a particularly useful nuclease for use in reducing run-to-run contamination when reusing a flowcell in a manner such as described with reference to FIGS. 1A-1B and 2, and/or when reusing a flowcell in a manner such as described with reference to FIGS. 3A-3B and 4.

### Example 2

A flowcell was prepared and used in the manner described with reference to FIGS. 3B and 4. In this example, the flowcell was based on the generation of a non-transformable surface, that is, surface primer anchor points that are not transformed by sequencing reagents and, thus, can be used after a sequencing run in a subsequent graft.

First, azides on a polymer surface were reduced to amines using the reducing agent THP. A methyltetrazine-PEG4-NHS ester was then attached. BCN-P5/P7 molecules were then grafted onto a portion of the resulting surface tetrazine sites, followed by standard clustering and sequencing. After each run, any remaining DNA or surface primers were digested using a nuclease. The used tetrazine sites on the polymer surface remained unusable. To reuse the flowcell, new BCN-P5/P7 were coupled to remaining tetrazine sites on the surface. After clustering and sequencing, the nuclease digest was performed using only MNase as the nuclease, after which new oligonucleotides were bound to remaining tetrazine sites on the surface using fresh BCN-P5/P7 molecules.

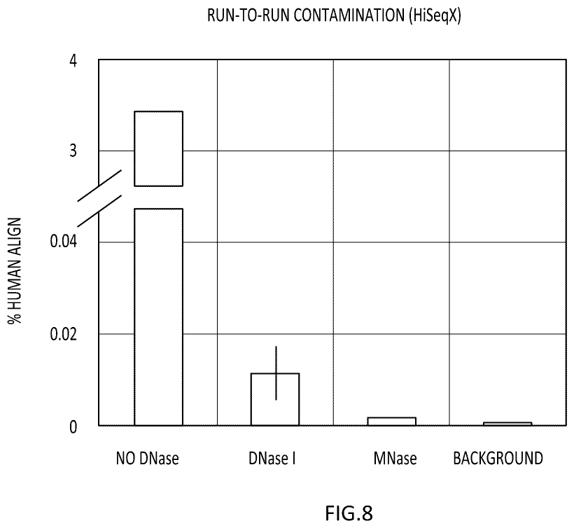

FIG. 8 illustrates data collected from using this example. The data illustrated in FIG. 8 are collected from 3 consecutive runs on a single flowcell using a NovaSeq instrument. As shown at plot A of FIG. 8, the overall intensity of the signal (C1) decreased with each run. This is expected because the number of tetrazine sites on the substrate decreased with each run. Nonetheless, the intensity of the signal remained sufficiently high to perform sequencing at each run, indicating that the flowcell satisfactorily may be reused at least three times. Additionally, as shown at plot B of FIG. 8, the data quality of the read (% Q30) was preserved between runs 1 and 2, but decreased with run 3. This is again expected because the number of tetrazine sites on the substrate decreased with each run. Nonetheless, the data quality remained sufficiently high to perform sequencing at each run, indicating that the flowcell satisfactorily may be reused at least three times.

As such, it is expected that flowcells prepared, used, and reused in accordance with 3A-3B and 4 may be reused for at least 2 times, or at least 3 times, or at least 5 times, or at least 10 times, or greater than 10 times, substantially without degradation in sequencing intensity, sequencing quality, and/or cluster monoclonality.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. An automated method conducted in a sequencing flowcell, the method comprising:
at a surface of the sequencing flowcell coupled to a first moiety, using a reagent to decouple a first complex from the first moiety, wherein the first complex comprises a second moiety which couples to the first moiety, and a polynucleotide coupled to the second moiety;
using a nuclease to digest polynucleotides in the sequencing flowcell; and
after using the reagent and after using the nuclease, coupling a second complex to the first moiety, wherein the second complex comprises a third moiety which couples with the first moiety, and an oligonucleotide coupled to the third moiety.

2. The automated method of claim 1, wherein the second moiety is coupled to the first moiety via a first non-covalent bond, and wherein the third moiety is coupled to the first moiety via a second non-covalent bond.

3. The automated method of claim 1, wherein the nuclease is used after the reagent.

4. The automated method of claim 1, wherein the reagent is used after the nuclease.

5. The automated method of claim 1, wherein the first complex comprises a protein with a first active site to which the oligonucleotide is coupled, and a second active site which corresponds to the second moiety.

6. The automated method of claim 1, wherein the surface is coupled to the first moiety using operations comprising:
reacting a fourth moiety at the surface with a fifth moiety of a molecule comprising the first moiety.

7. The automated method of claim 6, wherein the molecule further comprises a linker disposed between the fifth moiety and the first moiety.

8. The automated method of claim 1, further comprising:
before using the reagent and before using the nuclease, sequencing the polynucleotide.

9. The automated method of claim 8, further comprising, after coupling the second complex to the first moiety, using the oligonucleotide to amplify a template polynucleotide, and sequencing the amplified template polynucleotide.

10. The automated method of claim 9, wherein the flowcell is washed after the second complex is coupled to the first moiety, and before the oligonucleotide is used to amplify the template polynucleotide, or wherein the flowcell is washed after the polynucleotide is sequenced, before using the reagent, before using the nuclease, and before the second complex is coupled to the first moiety.

11. (canceled)

12. The automated method of claim 9, wherein the flowcell is washed after the polynucleotide is sequenced, after using the reagent, after using the nuclease, and before the second complex is coupled to the first moiety.

13. A kit for reusing a flowcell comprising a surface, the kit comprising:
a plurality of complexes, each of the complexes comprising a second moiety which can couple to a first moiety coupled to the flowcell surface, and a polynucleotide coupled to the second moiety;
a reagent to decouple the first and second moieties from one another; and
at least one nuclease to digest polynucleotides.

14. An automated method conducted in a sequencing flowcell, the method comprising:
at a surface of the sequencing flowcell coupled to a first moiety to which a polynucleotide is coupled, digesting the polynucleotide using Micrococcal Nuclease (MNase); and
after digesting the polynucleotide, coupling an oligonucleotide to a second moiety coupled to the surface.

15. The automated method of claim 14, wherein the polynucleotide is digested without use of a nuclease besides MNase.

16. The automated method of claim 14, wherein the polynucleotide is coupled to the first moiety via a first covalent bond, and wherein the oligonucleotide is coupled to the second moiety via a second covalent bond.

17. The automated method of claim 14, wherein the oligonucleotide is coupled to the second moiety using operations comprising:
reacting the second moiety with a third moiety of a molecule that comprises the oligonucleotide.

18. The automated method of claim 14, wherein the surface is coupled to the first moiety using operations comprising:
reacting a fourth moiety at the surface with a fifth moiety of a molecule comprising the first moiety.

19. The automated method of claim 18, wherein the molecule further comprises a linker disposed between the fifth moiety and the first moiety.

20. The automated method of claim 14, further comprising:
before using the MNase, sequencing the polynucleotide.

21. The automated method of claim 20, further comprising, after coupling the oligonucleotide to the second moiety, using the oligonucleotide to amplify a template polynucleotide, and sequencing the amplified template polynucleotide.

22. The automated method of claim 21, wherein the flowcell is washed after the oligonucleotide is coupled to the second moiety, and before the oligonucleotide is used to amplify the template polynucleotide, or wherein the flowcell is washed after the polynucleotide is sequenced, before using the MNase, and before the oligonucleotide is coupled to the second moiety.

23-26. (canceled)
